(19) United States
(12) Patent Application Publication
(10) Pub. No.: US 2007/0220636 A1
(43) Pub. Date: Sep. 20, 2007

(54) Watermelon Varieties Having Altered Sugar Ratios

(76) Inventors: Shmuel Wolf, Mazkeret Batya (IL); Amit Hotzev, Nirit (IL)

Correspondence Address: FENNEMORE CRAIG, 3003 NORTH CENTRAL AVENUE, SUITE 2600, PHOENIX, AZ, 85012, US

(21) Appl. No.: 10/588,410

(22) PCT Filed: Feb. 5, 2004
(86) PCT No.: PCT/IL04/00122

§ 371 Date: Mar. 12, 2007

(51) Int. Cl. A01H 5/08

(52) U.S. Cl. 800/300; 800/301; 800/302; 800/308

(57) ABSTRACT

The present invention relates to watermelon varieties producing fruit with altered sugar ratios having superior taste characteristics. Specifically, the present invention provides watermelon varieties producing fruit having fructose content of at least 50% of the total soluble sugar; varieties producing fruit having sucrose content of at least 65% of the total soluble sugar; and varieties producing fruit having a combined fructose and sucrose content of at least 90% of the total soluble sugar. The present invention further relates to the use of said varieties for the production of watermelons for the fresh and processed fruit market.

### FIELD OF THE INVENTION

The present invention relates to watermelon varieties producing fruit with altered sugar ratios having superior taste characteristics. Specifically, the present invention provides watermelon varieties producing fruit having fructose content of at least 50% of the total soluble sugar; varieties producing fruit having sucrose content of at least 65% of the total soluble sugar; and varieties producing fruit having a combined fructose and sucrose content of at least 90% of the total soluble sugar. The present invention further relates to the use of said varieties for the production of watermelons for the fresh and processed fruit market.

### BACKGROUND OF THE INVENTION

The edible quality of watermelon fruit depends upon the fruit texture, juiciness, color, and sweetness. Firm fruit, deep red in color and sweet in taste is the desired product, with high sugar content being perhaps the most important factor in ensuring consumer acceptability. Watermelon sweetness can be expressed as a function of the additive concentrations of the soluble sugars, fructose, glucose and sucrose (Kano, Y. 1991. Changes of sugar kind and its content in the fruit of watermelon during its development and after harvest. Env. Cont. Biol. 29, 159-166; Motsenbocker, C. E. and Picha D. H. 1995. Quality parameters of triploid watermelons. J. Veg. Crop. Prod. 2, 3-14). The ratio of these soluble sugars in known commercial cultivars of watermelon (Citrulus lanatus) is variable, however a typical ratio found is sucrose content in the range of 20-45%; glucose content in the range of 15-35%; and fructose content in the range of 20-40% of the total soluble sugar. Perception of sweetness is stimulated differently by each of these sugars and varies with temperature and concentration. In ascending order, glucose has the least effect on perceptible sweetness, followed by sucrose and fructose.

Very few studies have been aimed at investigating the factors affecting sugar level in watermelon fruit and the relationship between sugar level and fruit taste. In these limited studies, positive correlation was found between sweetness and total sugar level. It is important to note that the variation found between different commercial varieties in primary sugar concentrations or total soluble solids content was relatively low. These results confirmed an earlier observation of very low polymorphism in cultivated watermelon varieties, which were all offspring of the cultivated watermelon Citrulus lanatus (Katzir, N. et al. 1996. Length polymorphism and homologies of microsatellites in several Cucurbitaceae species. Theor. Appl. Gent. 93, 1282-1290; Lee, S. J. et al. 1996. Detection of genetic diversity using RAPD-OCR and sugar analysis in watermelon (Citrulus lanatus (Thumb.) Mansf) germplasm. Theor. Appl. Genet. 92, 719-725). The two wild types of the genus Citrulus—C. ecirrhous and C. colocynthis—may constitute a potential source of desirable traits, and are crossable with the cultivated watermelon (C. lanatus). The fruit of the wild species is characterized by white flesh and an extremely bitter taste (Navot N. et al. 1990. Linkage relationships of genes affecting bitterness and flesh color in watermelon. J. Heredity 81, 162-164).

The development of a commercial, superior watermelon variety requires a significant breeding effort. Specifically, it is necessary to break the linkage between the desirable traits and the bitterness of wild type species or other undesirable traits of wild type plants. The method chosen for breeding or selection depends on the mode of plant reproduction, the heritability of the trait(s) being improved, and the cultivar (i.e. variety) to be developed commercially (e.g. F₁ hybrid, or an open-pollinated variety). The complexity of the inheritance influences the choice of breeding method. One simple method of identifying a superior plant is to observe its performance relative to other experimental plants or to a widely grown standard cultivar, and to observe its performance in hybrid combinations with other plants. If single observations are inconclusive for establishing distinctness, observations in multiple locations and seasons provide a better estimate of its genetic worth. Proper testing and evaluation should detect any major faults and establish the level of superiority or improvement over current cultivars.

The development of commercial watermelon hybrids requires the development of homozygous stable parental lines. In breeding programs desirable traits from two or more germplasm sources or gene pools are combined to develop superior breeding varieties. Desirable inbred or parent lines are developed by continuous selfing and selection of the best breeding lines, sometimes utilizing molecular markers to speed up the selection process.

Once the parent lines that give the best hybrid performance have been identified, the hybrid seed can be produced indefinitely, as long as the homogeneity and the homozygosity of the parents is maintained. A single-cross hybrid is produced when two parent lines are crossed to produce the F₁ progeny. Much of the hybrid vigor exhibited by F₁ hybrids is lost in the next generation (F₂). Consequently, seed harvested from hybrid varieties is not used for planting stock.

Hitherto, attempts were made to obtain a sweeter watermelon by elevating the total sugar content in the fruit. However, such high content of sugar is not desired in terms of balanced diet, specifically as people tend to consume large portions of watermelon per serving.

Thus, it would be highly advantageous to have watermelon varieties producing fruits that provide all the characteristics of the market demand, which are sweeter, yet contain the same total amount or even fewer calories compared to currently available watermelon fruit.

### SUMMARY OF THE INVENTION

The present invention relates to robust watermelon varieties producing fruit with altered sugar ratios having superior sweet taste characteristics and the same total or lower calorie content compared to commercially available varieties, for use as fresh produce or for processed watermelon products. Specifically, the present invention provides watermelon varieties wherein the fruit produced by said varieties comprise an average fructose content of at least 50%; or an average sucrose content of at least 65%; or combined fructose and sucrose content of at least 90% of the total soluble sugar. The present invention further relates to seeds of the varieties of the present invention, to plants grown from the seeds, to their progeny, to fruit produced by the plants, to plant parts derived therefrom and to methods of producing these varieties. The present invention also relates to products obtained from the superior sweet watermelon fruit produced by the varieties of the present invention.

According to one aspect, the present invention provides robust watermelon plant varieties producing fruit with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation.

According to one embodiment, the present invention provides robust watermelon varieties producing fruit comprising an average fructose content of at least 50%, preferably 55%, more preferably 60% of the total soluble sugar.

According to another embodiment, the present invention provides robust watermelon varieties producing fruit comprising an average sucrose content of at least 65%, preferably 70%, more preferably 75% of the total soluble sugar.

According to yet another embodiment, the present invention provides robust watermelon varieties producing fruit wherein sucrose and fructose together comprise an average of at least 90%, preferably 95% of the total soluble sugar.

As used herein, the percentage of a specific sugar (sucrose or fructose) out of the total soluble sugar refers to an average ratio calculated based on the average sugar content obtained in a fruit crop. It is to be understood that the sugar content vary greatly depending on the stage of ripening, the conditions of cultivation, the measuring methods used and additional factors. The average value is obtained by measuring the sugar content of the crop at its peak sugar production, namely consisting of the ripe watermelon obtained by stress free cultivation, and by samples collected from the middle part (2-4 cm from the center) of cut watermelon fruits. Accordingly, as used herein, the term “average” refers to the mean plus or minus standard deviation.

As used herein, a fruit crop refers to the crop of a single plant, or preferably, to the fruit crop obtained from watermelon plants grown in a commercial scale.

As used herein, the term “superior sweet taste characteristics” refers to a watermelon fruit having an average content of at least 50%, preferably 55%, more preferably 60% fructose of the total soluble sugar; or at least 65%, preferably 70%, more preferably 75% sucrose of the total soluble sugar; or fruits wherein the combined content of fructose and sucrose comprise an average of at least 90%, preferably 95% of the total soluble sugar, wherein the fruits are devoid of the bitterness of the wild type Citrulus species and having a superior sweet taste. The term “altered sugar ratio” refers to the above described sugar ratios, which are altered compared to the hitherto known ratios of sugars in watermelon fruit, comprising sucrose content of 20-45%; glucose content of 15-35%; and fructose content of 20-40% of the total soluble sugar.

According to one embodiment, the watermelon varieties of the present invention are parental inbred lines.

According to another embodiment, the watermelon varieties of the present invention are hybrid varieties.

Within the scope of the present invention the term hybrid varieties encompasses any robust hybrid variety producing fruit with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation. Specifically the hybrid fruit comprise an average fructose content of at least 50% or an average sucrose content of at least 65% of the total soluble combined sugar, or wherein the average content of fructose and sucrose at least 90% of the total soluble sugar. The hybrid varieties advantageously can further comprise beneficial agronomical traits as are well known in the art including but not limited to high germination rate, herbicide resistance, insect resistance, resistance to bacterial, fungal or viral disease, resistance to various types of non-biotic stress, male sterility and improved nutritional value. The F₁ hybrid varieties of the present invention are superior over the parent lines in their plant vigor and adaptation for growth on a commercial scale. Specifically, the hybrid varieties of the present invention are firm and do not break open easily. Typically, fruit with high sugar content tend to break spontaneously upon maturation. The total sugar content of the varieties of the present invention can be kept lower, thus spontaneous breakage does not occur, while the sweet taste is maintained due to the unique sugar composition. The hybrid of the present invention can be a triploid, or an open-pollinated diploid.

The varieties of the invention are preferably non-genetically modified (non-GMO), however it is to be understood that the addition or deletion of traits by transformation is explicitly encompassed within the scope of the invention.

According to another embodiment, the present invention provides seeds of robust watermelon varieties, wherein the plants grown from the seeds or parts thereof produce fruit having superior sweet taste characteristic comprising an average fructose content of at least 50%, preferably 55%, more preferably 60% of the total soluble sugar.

According to yet another embodiment, the present invention provides seeds of robust watermelon varieties, wherein the plants grown from the seeds or part thereof produce fruit having superior sweet taste characteristic comprising an average sucrose content of at least 65% preferably 70%, more preferably 75% of the total soluble sugar.

According to further embodiment, the present invention provides seeds of robust watermelon varieties, wherein the plants grown from the seeds or part thereof produce fruit having superior sweet taste characteristic wherein fructose and sucrose together comprise an average of at least 90%, preferably 95% of the total soluble sugar.

The watermelon plants grown from the seeds of the present invention can be inbred parent lines or hybrid F₁ varieties.

Pollen and ovules from these watermelon plants; the seeds produced from same and the plants grown from the seeds; fruit produced by these plants; plants regenerated form tissue cultures regenerated from the plants of the present invention; and plants or parts thereof having all of the physiological and morphological characteristics of the watermelon plants of the present invention are also encompassed within the scope of the present invention.

According to one embodiment, the present invention provides a tissue culture regenerated from the watermelon plants of the present invention, wherein the tissue culture comprises cells or protoplasts from a tissue selected from the group consisting of leaves, pollen, embryos, roots, root tips, anthers, flowers, fruit and seeds.

According to one embodiment, the average fructose content in a commercial crop yield of the varieties of the present invention is at least 50% of the total soluble sugar; or at least 65% of the total soluble sugar; or the average content of fructose and sucrose together is at least 90% of the total soluble sugar, the fruit being devoid of bitterness and having superior sweet taste characteristics compared to currently available varieties. As described herein above, these values vary greatly depending on the conditions of cultivation, and other factors, and therefore represent mean plus minus standard deviation.

Hitherto, such high content of fructose, sucrose or their combination was not reported in watermelon fruit or in any cucurbit fruit, except wild type varieties not suitable for human consumption. These wild type varieties have extremely bitter taste, and a very small size. The novel varieties of the present invention including parental lines or hybrids adapted for commercial cultivation produce commercial scale crop yields with altered sugar ratios having equal or reduced total sugar content. The novel varieties are selected form varieties producing fruit having average fructose content of at least 50%, preferably 55%, more preferably 60% of the total soluble sugar; varieties producing fruit having average sucrose content reaches at least 65%, preferably 70%, more preferably 75% of the total soluble sugar; and varieties producing fruit an average content of fructose and sucrose together of at least 90%, preferably 95% of the total soluble sugar.

The varieties according to the present invention have a superior sweet taste, and are essentially devoid of the bitterness of the wild type. As described above, sucrose and fructose, specifically fructose, contribute the major portion to the sense of sweet taste. Common methods utilized previously to produce sweet watermelon fruit aimed at increasing the overall content of soluble sugar. Although the fruit obtained were indeed very sweet, they were also high in calories. The watermelon fruit of the present invention comprise the same calorie content of known commercial varieties while having sweeter taste, or comprise a lower calorie content while having the same sweet taste as in known commercial varieties. One gram of glucose, fructose or sucrose provides about 4 calories. Typically, the total sugar content of known commercial watermelon varieties is in the range of 8-15%, thus a serving size of about 200 grams comprises about 64-120 calories of soluble sugar. According to one embodiment, the fructose-containing watermelon fruit of the present invention comprise 20-40% less total sugar content, therefore 20-40% less calories.

According to another aspect, the present invention provides watermelon fruit with altered sugar ratios selected from at least one of elevated fructose and elevated sucrose content, having equal or reduced total sugar content, being devoid of bitterness and having superior sweet taste characteristics compared to currently available varieties. The fruit can be marketed as a fresh product or can serve as a source for processed watermelon products.

According to one embodiment, the average fructose content of the fruits is at least 50%, preferably 55%, more preferably 60% or higher of the total soluble sugar.

According to another embodiment, the average sucrose content of the fruits is at least 65%, preferably 70%, more preferably 75% or higher of the total soluble sugar.

According to yet another embodiment, the average combined fructose and sucrose content of the fruits is at least 90%, preferably 95% or higher of the total soluble sugar.

According to another aspect, the present invention provides a method for breeding watermelon varieties producing fruit having superior sweet taste characteristics compared to currently available varieties, suitable for commercial scale cultivation.

According to one embodiment, the present invention provides a method for breeding watermelon plants that produce watermelon fruit with altered sugar ratios selected from at least one of elevated fructose and elevated sucrose content, having equal or reduced total sugar content, being devoid of bitterness and having superior sweet taste characteristics compared to currently available varieties and suitable for commercial scale cultivation, including the steps of, crossing at least one wild type Citrulus species with a Citrulus lanatus to produce hybrid seeds, collecting the hybrid (F₁) seeds, growing plants from the F₁ seeds, pollinating the F₁ plants, collecting the hybrid seeds produced by the F₁ plants, growing plants from the seeds produced by the F₁ plants, measuring the total soluble sugar content of ripe fruit produced from the plants grown from the seeds of the F₁ plants; and selecting plants with watermelon fruit having desired characteristics including an average fructose content of at least 50%; or sucrose content of at least 65%; or fructose and sucrose content of least 90% of the total soluble sugar while devoid of the bitterness of the wild type Citrulus species.

According to one embodiment the step of pollinating the F₁ plants includes self pollination.

According to another embodiment, the step of pollinating the F₁ plants includes back crossing with a C. lanatus plant.

According to one preferred embodiment of the present invention the steps of crossing and selecting are repeated at least once.

According to another preferred embodiment of the present invention the method for breeding watermelon plants producing fruit having superior sweet taste characteristic additionally includes the steps of selfing, at least once, the selected plants, and further selecting plants with watermelon fruits having desired characteristics including an average fructose content of at least 50%; or sucrose content of at least 65%; or fructose and sucrose content of at least 90% of the total soluble sugar, being devoid of the bitterness of the wild type Citrulus to obtain watermelon advanced lines having a superior sweet taste characteristics.

As used herein, an advanced watermelon line of the present invention refers to a breeding line that already produce fruit comprising the desired sugar profile, and is devoid of the bitterness of the wild type, however may be subjected to further selection processes to produce stable, inbred parent lines.

According to yet another preferred embodiment of the present invention the method for breeding watermelon inbred lines producing fruit having superior taste characteristics additionally includes the steps of crossing a plant of advanced line with a Citrulus plant, selecting plants with watermelon fruits having desired characteristics including an average fructose content of at least 50%; or sucrose content of at least 65%; or combination of fructose and sucrose content of at least 90% of the total soluble sugar, and selfing the selected plants at least once to obtain watermelon inbred lines producing fruit with altered sugar ratios selected from at least one of elevated fructose and elevated sucrose content, having equal or reduced total sugar content, being devoid of bitterness and having superior sweet taste characteristics compared to currently available varieties and suitable for commercial scale cultivation.

According to one embodiment, the selfing is repeated from 1 to 12 times.

According to one embodiment, the Citrulus plant is a C. lanatus plant suitable for growth in commercial scale.

According to another embodiment, the Citrulus plant is a plant of an advanced watermelon line as defined herein above.

According to another aspect, the present invention provides a method for producing first generation (F₁) hybrid watermelon seeds from the plants varieties of the present invention.

According to one embodiment, the present invention provides a method for producing first generation hybrid seeds comprising crossing a first parent watermelon plant with a second parent watermelon plant and harvesting the resultant hybrid F₁ seeds, wherein the first and the second parent plants are inbred lines producing fruits with altered sugar ratios selected from at least one of elevated fructose and elevated sucrose content, having equal or reduced total sugar content, being devoid of bitterness and having superior sweet taste characteristics compared to currently available varieties, and are suitable for commercial scale cultivation.

According to yet another aspect, the present invention provides a method for producing watermelon plants using the varieties of the present invention, including progeny of the F₁ through F₇ breeding lines and backcrosses thereof.

According to one embodiment, the present invention provides a method of producing a watermelon plant derived from a watermelon line producing fruits having superior sweet taste characteristics according to the present invention. The first step of the method involves crossing a first watermelon plant line producing fruit with altered sugar ratios selected from at least one of elevated fructose and elevated sucrose content according to the present invention with a second watermelon plant to obtain F₁ progeny seed; the second step involves growing the F₁ progeny seed under suitable plant growth conditions to yield an F₁ watermelon plant of the first hybrid plant; optionally crossing the plant obtained in the second step with itself or with a third watermelon plant to yield second progeny seeds derived from said first hybrid plant and growing the second progeny seed under suitable plant growth conditions to yield additional watermelon plant derived of said first hybrid plant; and further optionally repeating the steps of crossing and growing from 1 to 7 or more times to generate further watermelon plants derived from the inbred line of the present invention, producing fruits with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation.

According to one embodiment, the first watermelon line from which the subsequent generations are derived produces fruit comprising an average fructose content of at least 50% of the total soluble content, being devoid of the bitterness of the wild type Citrulus species.

According to another embodiment, the first watermelon from which the subsequence generations are derived produces fruit comprising an average sucrose content of at least 65% of the total soluble content, being devoid of the bitterness of the wild type Citrulus species.

According to yet another embodiment, the first watermelon from which the subsequence generations are derived produces fruit comprising an average content of the combination of fructose and sucrose of at least 90% of the total soluble content, being devoid of the bitterness of the wild type Citrulus species.

According to another embodiment, the present invention provides plants derived from a plant produced according to the method described above, wherein the derived plant produces fruit with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation.

According to yet another embodiment, the present invention provides robust watermelon varieties according to the present invention, wherein the plants or progeny or parts thereof have been transformed so that its genetic material contains one or more transgenes operably linked to one or more regulatory elements. Watermelon plants and parts thereof produced from the transformed varieties are also encompassed within the scope of the present invention. According to one embodiment, the transformed gene or genes confer a characteristic selected from the group consisting of herbicide resistance, insect resistance, resistance to bacterial, fungal or viral disease, male sterility and improved nutritional value.

The present invention is explained in greater detail in the description, figures and claims below.

### BRIEF DESCRIPTION OF THE FIGURES

FIG. 1 Describes sugar content in wild type and commercial varieties of watermelon (Hybrid 313, Malali, Sugar baby). (A) Sucrose content (B) Fructose content (C) glucose content.

### DETAILED DESCRIPTION OF THE INVENTION

The present invention relates to the ongoing need for superior commercial cultivars to meet with market requirements. Such requirements cover a wide area of interest including cultivars with better crop yield, better taste, improved nutritional value, improved appearance and more.

Particularly, the present invention relates to hardy watermelon varieties, which produce fruits with superior taste characteristics, specifically a very sweet taste, while being low in calories. The present invention relates to such watermelon inbred lines, robust hybrids, fruits and seeds produced by same and progeny thereof.

According to one aspect, the present invention provides robust watermelon plant varieties producing fruit with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation.

The monosaccharides glucose and fructose and the disaccharide sucrose (composed of equal amounts of glucose and fructose) are the major soluble sugars founds within the flesh of many fruits. In terms of contribution to the sense of sweet taste, fructose is the most sweet sugar. In a scale where sucrose sweetness is given a value of 1, glucose value is 0.6-0.75 and fructose value is 1.6-1.8. Therefore, the sense of sweet taste depends not only on the total sugar content but also on the specific ratio between different sugar types. Typically, the ratio of soluble sugar in watermelon fruit of known commercial varieties comprise 20-45% sucrose; 15-35% glucose; and 20-40% fructose. The varieties of the present invention originated from crosses between wild type Citrulus species and commercial varieties of Citrulus lanatus. The wild type species produce fruit characterized, among other traits, as being extremely bitter. The present invention now provides watermelon varieties producing fruits with altered sugar ratios, which are completely devoid of the bitterness of the wild type species, having superiors sweet taste characteristics.

According to one embodiment, the present invention provides robust watermelon varieties producing fruit comprising an average fructose content of at least 50%, preferably 55%, more preferably 60% of the total soluble sugar.

According to another embodiment, the present invention provides robust watermelon varieties producing fruit comprising an average sucrose content of at least 65%, preferably 70%, more preferably 75% of the total soluble sugar.

According to yet another embodiment, the present invention provides robust watermelon varieties producing fruit wherein sucrose and fructose together comprise an average of 90% preferably 95% of the total soluble sugar.

Total soluble sugar content in watermelon fruit can be estimated by measuring the total soluble content (BRIX) using refractometer. Accurate sugar profile is obtained using HPLC. It is to be understood that the sugar profile of the watermelon varieties according to the present invention represents the average amount of any specific sugar within a fruit crop produced by these varieties. The fruit crop may refer to fruit produced by a single plant, or, preferably, to the fruit crop produced by plant grown on a commercial scale. Thus, as used herein in the specification and in the claims section that follows, an average fructose content of at least 50% of the total soluble sugar, for example, represent the mean plus or minus standard deviation of the fructose content measured for a ripe watermelon crop obtained by stress free cultivation, at its peak sugar production. The sugar profile of the watermelon fruit is also influenced by the sampling method employed. As exemplified herein below, a set of experiments was performed to establish a sampling method providing minimum variation between samples. Parameters examined by this set of experiments included stage of fruit ripening; method of samples collection; sampling region within the fruit. As used herein, average sugar content refers to an average measured in samples collected from the middle part of a cut fruit (2-4 cm from the center).

According to yet another embodiment, the watermelon varieties of the present invention are parental inbred lines.

According to a further embodiment, the watermelon varieties of the present invention are hybrid varieties.

As defined herein, parent lines refers to open pollinated, inbred lines, stable for the desired traits over cycles of self-pollination and planting. The parent lines of the present invention were developed from a cross between a wild type Citrulus species and a Citrulus lanatus plant. Among others, Citrulus colocynthis served as the wild type parent. Citrulus lanatus plants used were selected from a germplasm collection of proprietary breeding material belonging to A.B. Seeds Ltd. an applicant of the present invention. High variation is found within wild type Citrulus species and therefore they can serve as a source for new traits. However, in regard with taste, the wild type varieties comprise certain substances that are extremely bitter.

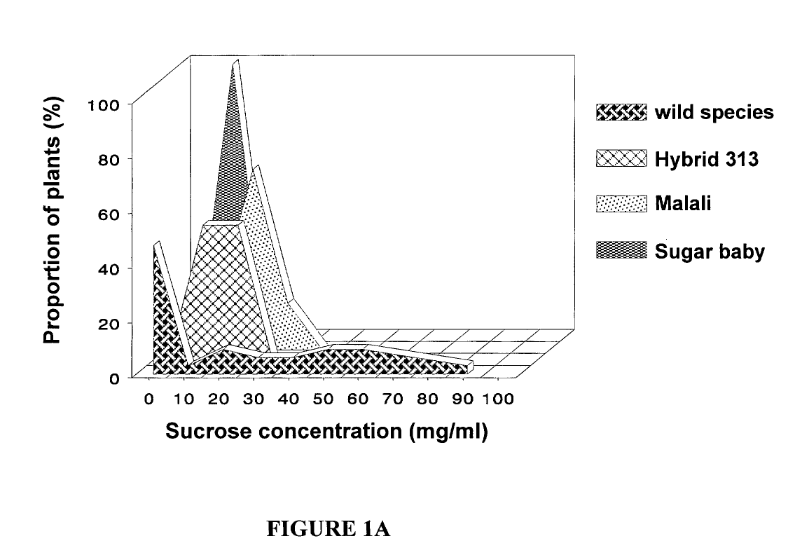

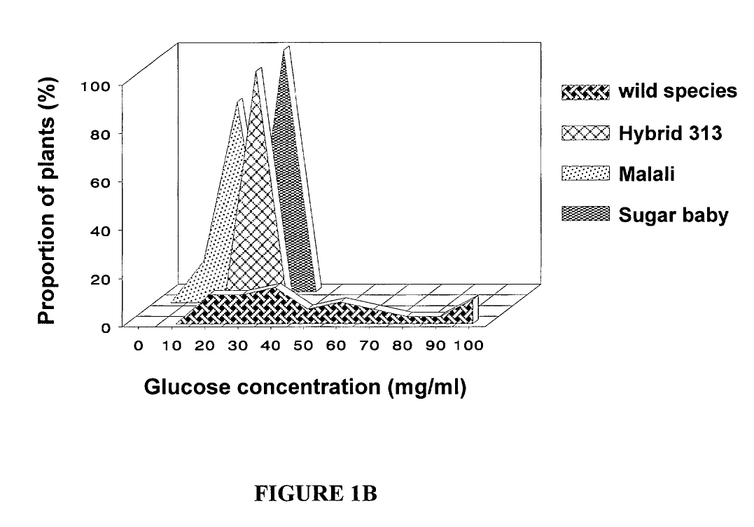

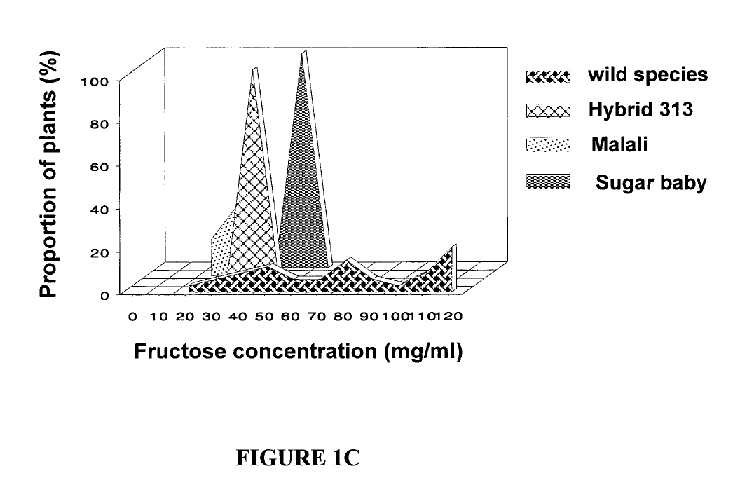

A survey of diversity within wild type germplasm of the genus Citrulus was performed. In this study, crosses were made between various wild species of Citrulus and several commercial varieties. All of these crosses were successful, yielding fertile F₁ offspring. Analysis of sugar content in almost 100 backcrossed (BC) lines between these hybrids and commercial varieties has indicated very high variation in both the level of total sugar and the proportions of the different soluble sugars. Examination of the different sugars showed that there were individuals exhibiting up to four times higher levels than the commercial varieties. Additionally, a wide variation in the proportions of the various sugars within this germplasm was also obtained, in contrast to the similar sugar profile obtained for commercial varieties derived from C. lanatus (FIG. 1). Such variation enabled the selection of genotypes with specific relationships between the different primary sugars. The F₁ hybrids obtained from the cross between the wild type Citrulus species and the commercial lines were left for self pollination, seeds were collected and sugar profile of the fruits and their taste was examined. Hybrids showing high sucrose or fructose proportion, and lower or no bitterness were selected, and their seeds were sown. Plants grown from these F₁ seeds were again left for self pollination. In addition, backcrosses (BC) were also made between selected progeny plants and either their parent lines or commercial parent line. Planting and selecting the best performing plants (plants producing fruit comprising high proportion of sucrose or fructose while devoid of the bitterness of the wild type species) was repeated for several times as exemplified herein below, to obtain stable parental lines producing fruits comprising an average fructose content of at least 50%; stable parental lines producing fruits comprising an average sucrose content of at least 65%; and stable parental lines producing fruits comprising an average of at least 90% of fructose and sucrose together, while devoid of the bitterness of the wild type species and having superior sweet taste characteristics.

The selection process optionally included the use of an isozyme marker to differ and remove plants producing fruits comprising the desired sugar profile, however having a very bitter taste. This isozyme, designated Pgm-1, is linked to the dominant gene Bi, which governs fruit bitterness (Navot, N. et al., supra). Plants showing the Pgm-1 isozyme on a starch gel were removed from further analyses.

According to one embodiment, the present invention provides a method for breeding watermelon plants that produce watermelon fruit with altered sugar ratios selected from at least one of elevated fructose and elevated sucrose content, having equal or reduced total sugar content, being devoid of bitterness and having superior sweet taste characteristics compared to currently available varieties and suitable for commercial scale cultivation, including the steps of: crossing at least one wild type Citrulus species with a Citrulus lanatus to produce hybrid seeds, collecting the hybrid (F₁) seeds, growing plants from the F₁ seeds, pollinating the F₁ plants, collecting the hybrid seeds produced by the F₁ plants, growing plants from the seeds produced by the F₁ plants, measuring the total soluble sugar content of ripe fruit produced from the plants grown from the seeds of the F₁ plants; and selecting plants with watermelon fruits having desired characteristics including an average fructose content of at least 50%; or sucrose content of at least 65%; or combination of fructose and sucrose content of least 90% of the total soluble sugar while devoid of the bitterness of the wild type Citrulus species.

According to one embodiment the step of pollinating the F₁ plants includes self pollination.

According to another embodiment, the step of pollinating the F₁ plants includes back crossing with a C. lanatus plant.

According to one preferred embodiment of the present invention the steps of crossing and selecting are repeated at least once.

According to another preferred embodiment of the present invention the method for breeding watermelon plants having superior sweet taste characteristic additionally includes the steps of selfing, at least once, the selected plants, and further selecting plants with watermelon fruits having desired characteristics including an average fructose content of at least 50%; or sucrose content of at least 65%; or combined fructose and sucrose content of at least 90% of the total soluble sugar, being devoid of the bitterness of the wild type Citrulus to obtain watermelon advanced lines having a superior sweet taste characteristics.

As exemplified herein below, the advanced lines of the present invention were F₈ to F₉ lines, selected for high sucrose content, while devoid bitter taste. These lines were used in backcrosses during the breeding process for producing the inbred lines of the present invention.

According to yet another preferred embodiment of the present invention the method for breeding watermelon inbred lines producing fruit having superior taste characteristics additionally includes the steps of crossing a plant of advanced line with a Citrulus plant, selecting plants with watermelon fruits having desired characteristics including an average fructose content of at least 50%; or sucrose content of at least 65%; or combination of fructose and sucrose content of at least 90% of the total soluble sugar, and selfing the selected plants at least once to obtain watermelon inbred lines with altered sugar ratios selected from at least one of elevated fructose and elevated sucrose content, having equal or reduced total sugar content, being devoid of bitterness and having superior sweet taste characteristics compared to currently available varieties and suitable for commercial scale cultivation.

According to one embodiment, the selfing is repeated from 1 to 12 times.

According to one embodiment, the Citrulus plant is a C. lanatus plant suitable for growth in commercial scale.

According to another embodiment, the Citrulus plant is a plant of an advanced watermelon line as defined herein above.

The obtained parental lines were crossed to obtain robust F₁ hybrids. The hybrids of the present invention, in addition to producing fruits with superior sweet taste characteristics, can further comprise other beneficial agronomic traits, including resistance to various diseases and non-biotic stresses. The F₁ hybrids can be indefinitely produced from the stable parent lines of the present invention. Specifically, the hybrid varieties of the present invention are firm and do not break open easily. Typically, fruit with high sugar content tend to break spontaneously upon maturation. The total sugar content of the varieties of the present invention can be kept lower, thus spontaneous breakage does not occur, while the sweet taste is maintained due to the unique sugar composition. The hybrid of the present invention can be a triploid, or an open-pollinated diploid.

According to one embodiment, the present invention provides a method for producing first generation hybrid seeds comprising crossing a first parent watermelon plant with a second parent watermelon plant and harvesting the resultant hybrid F₁ seeds, wherein the first and the second parent plants are inbred lines producing fruits with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation.

According to another embodiment, the present invention also provides a first generation F₁ hybrid watermelon plants that are produced by growing the hybrid watermelon seeds produced by the above-described method.

According to yet another aspect, the present invention provides a method for producing watermelon plants using the varieties of the present invention, including progeny of the F₁ through F₇ breeding lines and backcrosses thereof.

The present invention also relates to seeds harvested on the F₁ hybrid watermelon plants and plants grown from these seeds. A common practice in plant breeding is using the method of backcrossing to develop new varieties by single trait conversion. The term single trait conversion as used herein refers to the incorporation of new single gene into a parent line wherein essentially all of the desired morphological and physiological characteristics of the parent lines are recovered in addition to the single gene transferred. The term backcrossing as used herein refers to the repeated crossing of a hybrid progeny back to one of the parental watermelon plants. The parental watermelon plant which contributes the gene for the desired characteristic is termed the non-recurrent or donor parent. This terminology refers to the fact that the non-recurrent parent is used one time in the backcross protocol and therefore does not recur. The parental watermelon plant to which the gene or genes from the non-recurrent parent are transferred is known as the recurrent parent as it is used for several rounds in the backcrossing protocol. In a typical backcross protocol, a plant from the original varieties of interest (recurrent parent) is crossed to a plant selected from second varieties (non-recurrent parent) that carries the single gene of interest to be transferred. The resulting progeny from this cross are then crossed again to the recurrent parent and the process is repeated until a watermelon plant is obtained wherein essentially all of the desired morphological and physiological characteristics of the recurrent parent are recovered in the converted plant, in addition to the single transferred gene from the non-recurrent parent. Backcrossing methods can be used with the present invention to improve or introduce a characteristic into the parent lines.

The present invention encompasses any part of the parent plant lines or of the hybrid plant, including pollen, ovules, fruits and tissue cultures regenerated from these plants. Pollen and ovules are used in breeding programs, in general and as described by the present invention. Tissue culture of watermelon can be used for the in vitro regeneration of a watermelon plant as is known in the art (Compton, M. E. 2000. Interaction between explant size and cultivar affects shoot organogenic competence of watermelon cotyledons. HortScience 35, 749-750; Compton, M. E. and Gray, -D. J. 1994. Adventitious shoot organogenesis and plant regeneration from cotyledons of tetraploid watermelon. HortScience. 29, 11-213).

Plants comprising within their pedigree a watermelon producing fruit having superior taste characteristics according to the present invention, and methods for producing same, are also encompassed within the scope of the present invention.

As used herein, the watermelon line from which the subsequence generations are derived produces fruit with altered sugar ratios selected from at least one of elevated fructose and elevated sucrose content, having equal or reduced total sugar content, being devoid of bitterness and having superior sweet taste characteristics compared to currently available varieties, and are suitable for commercial scale cultivation.

According to one embodiment, the present invention provides a method of producing a watermelon plant derived from a watermelon line producing fruits having superior sweet taste characteristics according to the present invention. The first step of the method involves crossing a first watermelon inbred line according to the present invention with a second watermelon plant to obtain F₁ progeny seed; the second step involves growing the F₁ progeny seed under suitable plant growth conditions to yield an F₁ watermelon plant of the first hybrid plant; optionally crossing the plant obtained in the second step with itself or with a third watermelon plant to yield second progeny seeds derived from said first hybrid plant and growing the second progeny seed under suitable plant growth conditions to yield additional watermelon plant derived of said first hybrid plant; and further optionally repeating the steps of crossing and growing from 1 to 7 or more times to generate further watermelon plants derived from the inbred line of the present invention.

According to yet another embodiment, the present invention provides robust watermelon varieties according to the present invention, wherein the plants or progeny or parts thereof have been transformed so that its genetic material contains one or more transgenes operably linked to one or more regulatory elements. Watermelon plants and parts thereof produced from the transformed varieties are also encompassed within the scope of the present invention. According to on embodiment, the transformed gene or genes confer a characteristic selected from the group consisting of herbicide resistance, insect resistance, resistance to bacterial, fungal or viral disease, male sterility and improved nutritional value.

### EXAMPLES

### Example 1

### Calibration of the Sampling Method

Extraction Method

To calibrate the sugar extraction protocol a comparison was made between samples crushed rigorously upon arrival to the laboratory and then exposed to ethanol extraction and samples stored in the freezer (−20° C.) for a few days and then thawed, crushed and filtered, such that sugar content was determined in the serum. All samples were diluted in water and filtered through a 0.45 μm membrane HPLC filter. Sugars were analyzed in an analytical HPLC system (Auto sampler 360, Pump System 320, Kontron instruments, Switzerland) fitted with a Sugar-Pak I column (6.5 mm×300 mm; Waters) using a reflective-index detector 475 (Kontron instruments, Switzerland). The column was heated to 80° C. The running solvent was HPLC grade water. Flow rate 0.5 ml/nim.

Analyses of the data obtained from these experiments revealed no significant difference between the two procedures. These results indicated that samples can be collected from the filed at a relatively short period of time and stored at −20° C. before subjected to sugar analyses.

Sampling Procedure

This set of experiments was aimed to define the variation between samples collected from different regions of the fruit.

Half fruits were divided to three sections: the outer, the middle and the center. For sugar analyses, three fresh fruit tissue were collected from the outer circle of each fruit and the center circle (inner part) of each fruit.

The levels of the various sugars were higher (about 15%) in the samples collected from the inner part of the fruit as compared with samples obtained from the outer circle. It is important to note that variation between sugar levels in samples collected from the same region (outer or center) was low (less than 10%).

Based on the data obtained from these experiments a sampling protocol was calibrated such that samples (3-5 gr) were collected from the middle part (2-4 cm from the center) of cut watermelon fruits.

### Example 2

### Sugar Levels in Fruits of Wide Germplasm Variety of Watermelon

Two field experiments were conducted to analyze sugar composition and content in a wide collection of watermelon germplasm.

Spring Field Trial

The spring experiment (1999) included 242 lines of wide collection of F₁ crosses between the wild species of Citrulus and commercial varieties. Each line was grown in three replicated plots of four plants in each plot (total of almost 3000 plants). The plants were grown in the field of Beit Elazari (Israel) under net screen. Plants were left for self pollination until fruit maturation, fruits were collected and sugar content was analyzed as described herein above. Total sugar content in the fruits ranged from very low (2-5%) to very high (25-27%) in the various lines.

Winter Field Trial

The winter experiment (2000) included 139 progeny lines that were selected based on the data obtained in the previous spring (1999). This experiment was conducted in a heated greenhouse in Beit Elazari. Sugar content was lower in the winter as compared to the spring trial. Nevertheless, the value of total sugars was found to be over 20% in several specific lines even at this season.

### Example 3

### Sugar Levels in Fruits of Advanced Watermelon Breeding Lines

In addition to the evaluation of the wide genetic germplasm, sugar content was determined in advanced breeding lines. The advanced breeding lines were F₈ to F₉ progenies of the hybrid plants described in example 2 above, that were selected in each generation for high sucrose content.

Two field trials were conducted during the summer and the autumn of 1999 in Beit Elazari (Israel). Values of sucrose for the various control lines (plot numbers 215, 216, 285-288) were in the range of 1-2% in this particular experiment. Sucrose content in most of the advanced lines was significantly higher, in some cases up to 5-6 fold higher compared to the controls. It is important to note that variation between fruit within the high sucrose advanced lines was relatively low.

### Example 4

### Breeding Process to Obtain Super Sweet Watermelon Fruit

A. Field Experiments Summer 2001. Sowing—April 2001; Harvest—July 2001

Seeds of F₁ plants described in example 2 above that showed high total sugar content with the desired sugar profile were collected and planted. 120 lines of generations F₁-F₄ were selected as potential candidates for the breeding program. Progeny of these lines, as well as back-crosses (BC) to selected advanced watermelon parents described in example 3 above, were grown in Bet Elazari. Sugar analysis was performed for fruit collected from about 500 plants from this population (F₂-F₅ generations as well as BC₁ and BC₂).

One of the most striking result obtained during this experiment was the high correlation coefficient found between sugar content and profile of the parent lines, (analyzed in 2000) and sugar content and profile in the progenies analyzed in 2001 (Table 1). The parents were selected according to the content of a specific sugar (Fructose or sucrose) with the aim to obtain pure lines characterized by a unique sugar composition. A significant positive correlation was found in all parameters: the content of each specific sugar and the proportion of the various sugars. The higher correlation coefficient found between the years 2000 and 2001, as compared with that obtained between the years 1999 and 2000, indicated that the selection based on HPLC sugar analyses was justified. Moreover, an evaluation of the F₅ progeny indicated significantly lower variation between individuals within a family.

It is important to note that in many cases the specific characteristic (e.g. high sucrose, high fructose concentrations), was found in crosses between the selected lines and different commercial varieties, indicating that a specific sugar composition can be easily combined into new hybrid varieties.

TABLE 1
Correlation analyses between the season of 2000 and 2001

| Parameter | Correlation coefficient |
|---|---|
| Total sugars | R = 0.17*** |
| Absolute sucrose concentration: | R = 0.44*** |
| Ratio of Sucrose/Total sugar | R = 0.50*** |
| Absolute fructose concentration: | R = 0.17*** |
| Ratio of Fructose/Total sugar | R = 0.49*** |
| Absolute glucose concentration: | R = 0.35*** |
| Ratio of Glucose/Total sugar | R = 0.38*** |

***P < 0.001

B. Field Experiments During the Year 2002

Following the field trials performed in the years 1999-2001 described above, over 100 lines of generations F5-F7 were selected as potential candidates for further breeding. Progeny of these lines, as well as backcrosses (BC) to selected advanced parent lines, were analyzed. Sugar analysis was performed for fruit collected from over 600 plants from this population (F6-F8 generations as well as back crosses to several promising lines).

Evaluation of the sugars analyses within this wide progeny indicated that variation in the content of a specific sugar was very low in F₇ and F₈ lines. Some variation was still found in F₆ lines.

Selection of Specific Lines:

Sugars content in the various control varieties (Crimson Sweet, Odem) were in the range of 8-9%. Out of these values, glucose and fructose comprised 30-40% each, while sucrose proportion was 15-30% of the total sugars.

High Sucrose Proportion

High sucrose proportion was measured in fruits of the following family lines: code# 740, 741, 742, 744; code# 768, 769; code# 772, 773, 774, 775; code# 784, 785, 787; and code# 796, 797. All the indicated lines were progenies of parents that were selected due to their high sucrose concentration.

High Fructose Proportion

High fructose proportion was measured in fruits of the following family lines: code# 701, 703, 705, 706, 707, 712, 717; code# 719, 720; code #722, 724, 725; code# 727; and code# 792. The variation in fructose level among fruit collected form different plants in each line was extremely low.

High Content of Total Sugar

Some of the aforementioned lines characterized by high content of specific sugar were also characterized by high content of total sugar (e.g. code# 701, 703, 705, 706, 707, 719, 720 (high fructose) and 773, 774, 775, 787, 796 (high sucrose).

Other lines such as code# 782, 783, 801 (relatively high combined content of sucrose and fructose), and specific individual plants in the lines code# 709, 744, 768, 769, 776, 786, 795, 797 were also characterized by very high content of total sugar in the fruit.

As mentioned previously, the specific characteristic (e.g. high sucrose, high fructose content) was found in progenies of crosses between the selected lines and different commercial varieties, indicating that a specific sugar composition can be easily combined into new hybrid varieties.

C. Field Experiments During the Year 2003

Following the previous field experiments, over 60 lines of generations F₆-F₇ were selected as potential candidates to continue with the breeding program. Progeny of these lines, as well as backcrosses (BC) to selected advanced parent lines, were analyzed in this experiment. The data include sugar analysis of fruits collected from over 500 plants from this population (F₇-F₈ generations as well as back crosses to several promising lines).

Evaluation of the sugar analyses within this population indicated that variation in the content of a specific sugar was very low in F₇ and F₈ lines. Most of these lines were almost homozygous for the respective character (content of specific sugar), and therefore can be designated as stable parent lines.

Selection of Specific Lines

Sugars content in the various control varieties (Crimson Sweet, Odem) were in the range of 8-9%. Out of these values, the proportion of sucrose was 30-40%, the proportion of glucose was in the range of 25-30%, and the proportion of fructose was 30-40% of the total sugars.

High Sucrose Proportion

High sucrose proportion was measured in fruit of the following family lines: code# 318 and 319 (selected from family #740), 321 and 322 (selected from family #742), 324 (selected from family #744), #330, #332-335 (selected from families #768 and 769, respectively), #339 and 340 (selected from family #773), #359 and 361 (selected from families #785 and 787, respectively).

It is important to note that all the indicated lines were progenies of parents that were selected due to their high sucrose content.

High Fructose Proportion

High fructose proportion were measured in fruit of the following family lines: code# 300-302 (selected from family #701), 303-305 (selected from family #705), 306-308 (selected from family #706), 311, 312, 314, (selected from family #711), 315 and 316 (selected from families #719 and 720, respectively). In all the families the average proportion of fructose was 50% or higher, wile in some individuals fructose concentration was over 60% of the total soluble sugar.

High Content of Total Soluble Sugar

Some of the aforementioned lines characterized by high content of a specific sugar, were also characterized by high content of total soluble sugar:

Code # 318 (selected from family #740 [high sucrose]), #324 and 325 (selected from family #744 [high sucrose]) and code #332.

Some lines were characterized by high level of total soluble sugar due to relatively high content of glucose. Code # 309 and 310 (selected from family #709), code #326 and 327, while some lines such as #338-342, had relatively high, combined content of fructose and sucrose.

Representative results from the experiments conducted during the years 2001 to 2003 are summarized for lines having high fructose proportion (Table 2) and high sucrose proportion (Table 3) below.

TABLE 2
Sugar content in lines selected for high fructose content

| | | | | | |
|---|---|---|---|---|---|
| Code | Sucrose | Glucose | Fructose | Total | Fructose/total |

2001

| | | | | | |
|---|---|---|---|---|---|
| 501-1 | 0.6 | 2.7 | 5.0 | 8.3 | 59.5 |
| 501-1 | 0.6 | 2.7 | 5.0 | 8.3 | 59.5 |
| 506-1 | 1.2 | 2.9 | 5.1 | 9.2 | 55.3 |
| 506-1 | 1.2 | 2.9 | 5.1 | 9.2 | 55.3 |
| 506-1 | 1.2 | 2.9 | 5.1 | 9.2 | 55.3 |
| 507-1 | 2.1 | 3.1 | 4.6 | 9.8 | 46.7 |
| 507-1 | 2.1 | 3.1 | 4.6 | 9.8 | 46.7 |

2002

| | | | | | |
|---|---|---|---|---|---|
| 701-4 | 1.8 | 6.7 | 10.8 | 19.3 | 56.1 |
| 701-5 | 2.8 | 6.1 | 11.0 | 19.9 | 55.2 |
| 705-2 | 5.6 | 4.3 | 9.4 | 19.3 | 48.8 |
| 705-5 | 1.8 | 5.0 | 10.7 | 17.5 | 61.4 |
| 705-6 | 1.5 | 6.7 | 10.6 | 18.8 | 56.5 |
| 706-1 | 0.8 | 6.1 | 10.5 | 17.4 | 60.4 |
| 706-4 | 2.0 | 5.9 | 10.8 | 18.8 | 57.7 |

2003

| | | | | | |
|---|---|---|---|---|---|
| 300-(1-6) | 0.0 | 4.2 | 5.6 | 9.8 | 57.3 |
| 301-(1-5) | 0.8 | 4.4 | 6.0 | 11.2 | 54.0 |
| 303-(1-6) | 2.3 | 3.1 | 5.3 | 10.7 | 49.1 |
| 304-(1-4) | 1.5 | 2.3 | 4.1 | 7.8 | 51.7 |
| 305-(1-6) | 1.6 | 2.4 | 4.3 | 8.3 | 51.5 |
| 306-(1-4) | 0.5 | 4.1 | 5.4 | 10.0 | 54.0 |
| 307-(1-6) | 0.2 | 3.9 | 5.4 | 9.5 | 56.9 |

TABLE 3
Sugar content in lines selected for high sucrose content

| | | | | | |
|---|---|---|---|---|---|
| Code | Sucrose | Glucose | Fructose | Total | Sucrose/total |

2001

| | | | | | |
|---|---|---|---|---|---|
| 547-5 | 9.8 | 1.7 | 4.3 | 15.8 | 62.1 |
| 558-2 | 7.5 | 1.9 | 4.6 | 13.9 | 53.7 |
| 574-5 | 5.3 | 0.9 | 2.2 | 8.4 | 62.8 |
| 574-5 | 5.3 | 0.9 | 2.2 | 8.4 | 62.8 |
| 577-1 | 6.5 | 3.1 | 5.2 | 14.8 | 44.0 |
| 577-1 | 6.5 | 3.1 | 5.2 | 14.8 | 44.0 |

2002

| | | | | | |
|---|---|---|---|---|---|
| 742-6 | 10.4 | 1.1 | 2.8 | 14.3 | 72.8 |
| 753-6 | 7.8 | 2.1 | 4.7 | 14.5 | 53.8 |
| 769-2 | 9.6 | 2.4 | 3.4 | 15.4 | 62.1 |
| 769-7 | 11.7 | 2.6 | 4.4 | 18.7 | 62.5 |
| 770-7 | 11.3 | 0.4 | 3.5 | 15.2 | 74.4 |
| 773-2 | 13.7 | 2.8 | 5.5 | 22.0 | 62.3 |
| 773-5 | 15.8 | 2.6 | 5.0 | 23.5 | 67.3 |

2003

| | | | | | |
|---|---|---|---|---|---|
| 321-(1-5) | 6.8 | 1.2 | 2.4 | 10.5 | 64.8 |
| 330-(1-6) | 7.2 | 1.2 | 3.4 | 11.8 | 61.1 |
| 334-(1-6) | 8.2 | 1.1 | 2.9 | 12.1 | 67.6 |
| 335-(1-6) | 8.1 | 0.8 | 2.1 | 11.0 | 73.6 |
| 337-(1-6) | 7.6 | 1.6 | 3.3 | 12.5 | 60.7 |
| 339-(1-6) | 9.0 | 1.9 | 2.9 | 13.8 | 65.4 |
| 340-(1-6) | 13.1 | 2.1 | 3.5 | 18.7 | 70.2 |

### Example 5

### Taste Evaluation

Taste evaluations were performed during the breeding program to select varieties comprising the desired altered sugar ratio while devoid of the bitter taste contributed by the wild type species. A simple ranking test was used for a comparison between the examined varieties and a control variety. Alternatively, a “Difference from control” test was applied, where the subjects had to rate the difference between the control and the test samples using the scale (−)3 to (+)3, where 0 is no difference between the two samples; (−)3 the examined sample is much lower than the control; (+)3 the examined sample is much higher than the control. The tests were conducted based on the detailed procedure in “Sensory Evaluation Techniques” (Meilgaard, M, Civille, G. V. and Carr, B. T eds., CRC press). The subjects (evaluators) were asked to rank the samples based on two criteria: sweetness and overall taste.

A first taste evaluation was performed in April 2001 using the simple ranking test. Fruits collected from four plant lines were evaluated by 30 subjects in two successive days.

The evaluated lines were:

A—Plant line #3001

B—Plant line #843

C—Plant line #2020

D—Plant line #313, a commercial control variety (the most popular seedless variety)

Plant lines 3001 (A) and 2020 (C) were ranked significantly higher than the other lines in both parameters (sweetness and overall taste). Similar ranking was found in two independent tests (15 and 16 of April). In this specific experiment, high significant difference was found in sucrose concentration between the four plant lines.

Correlation analyses between taste/sweetness and the content of each sugar, the proportion of the various sugars and the level of total sugars indicated high correlation coefficient between the proportion of sucrose and taste (R=0.77; P<0.001) and between the proportion of sucrose and sweetness (R=0.75; P<0.001).

Second taste evaluation was performed in July 2001, examining the same plant lines after further breeding. Seven fruit from each of the line examined were tested. 105 subjects participated in the test during 7 days.

At this stage, the variation in sugar level and composition between the examined plant lines was shown to be relatively low. The overall score indicated no significant differences in sweetness or overall taste between the four lines. These results indicate that the lines are already devoid of the bitterness of the wild type, as they were as sweet and tasty as the widely used line used as a control. However, no significant correlation was obtained between taste, sweetness and sugar content or ratio, indicating that further selection is required to obtain stable characteristics. Nevertheless, it is interesting to note that plant line #313 had the highest score in sweetens and overall taste, and fruit of this plant line had the highest sucrose concentration as well as total sugars.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1-54. (canceled)

55. A robust watermelon variety producing fruit with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation.

56. The watermelon variety of claim 55, wherein the average fructose content is at least 50% or at least 55% of the total soluble sugar.

57. The watermelon variety of claim 55, wherein the average fructose content is at least 60% or at least 65% of the total soluble sugar.

58. The watermelon variety of claim 55, wherein the average sucrose content is at least 70% or at least 75% of the total soluble sugar.

59. The watermelon variety of claim 55, wherein the average combined content of fructose and sucrose is at least 90% or at least 95% of the total soluble sugar.

60. The variety of claim 55, wherein the variety is an inbred parent line.

61. The variety of claim 55, wherein the variety is a hybrid.

62. A watermelon fruit produced from the watermelon variety of claim 55.

63. A seed of a robust watermelon variety, wherein a plant grown from the seed is a watermelon variety of claim 55.

64. A watermelon plant, or part thereof, produced by growing the seed of claim 63.

65. The watermelon plant, or part thereof of claim 64, wherein the part thereof is a pollen grain, an ovule, or tissue culture of regenerable cells of the plant.

66. The plant or part thereof of claim 64, further comprising at least one additional trait selected from the group consisting of herbicide resistance, insect resistance, resistance to bacterial, fungal or viral disease, male sterility and improved nutritional value.

67. The plant or part thereof of claim 66, further comprising at least one additional trait selected from at least one type of disease resistance and at least one type of stress resistance.

68. The plant or part thereof of claim 66, wherein the additional trait is introduced by breeding, single trait conversion, or transformation.

69. The plant, or part thereof, of claim 64, wherein the plant or part thereof is transgenic and contains one or more transgenes operably linked to one or more regulatory elements.

70. The tissue culture according to claim 65, comprising cells or protoplasts from a tissue selected from the group consisting of leaves, pollen, ovules embryos, roots, root tips, anthers, flowers, fruit and seeds.

71. The tissue culture of regenerable cells of claim 70, wherein the tissue regenerates plants producing fruit with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation.

72. A watermelon plant regenerated from the tissue culture of claim 71.

73. A method for breeding a watermelon plant producing fruit with altered sugar ratios selected from at least one of elevated fructose and elevated sucrose content, having equal or reduced total sugar content, being devoid of bitterness and having superior sweet taste characteristics compared to currently available varieties and suitable for commercial scale cultivation, comprising the steps of:
a. crossing at least one wild type Citrulus species with a Citrulus lanatus to produce F₁ hybrid seeds;
b. collecting the hybrid F₁ seeds;
c. growing plants from the F₁ seeds;
d. pollinating the F₁ plants;
e. collecting the hybrid seeds produced by the F₁ plants;
f. growing plants from the seeds produced by the F₁ plants;
g. measuring the total soluble sugar content of ripe fruit produced from the plants grown from the seeds of the F₁ plants; and
h. selecting plants with watermelon fruit comprising an average fructose content of at least 50%; sucrose content of at least 65%; or fructose and sucrose content of least 90% of the total soluble sugar being devoid of the bitterness of the wild type Citrulus species.

74. The method of claim 73, wherein the pollination in step (d) includes self pollination or back crossing with a C. lanatus plant.

75. The method of claim 73, wherein the steps of crossing and selecting are repeated at least once.

76. The method of claim 73, further comprising the step of selfing, at least once, the selected plants, and further selecting plants producing fruit comprising an average fructose content of at least 50%; or sucrose content of at least 65%; or fructose and sucrose content of at least 90% of the total soluble sugar being devoid of the bitterness of the wild type Citrulus, to obtain super sweet watermelon advanced lines.

77. The method of claim 76, further comprising the steps of:
a. crossing a Citrulus advanced line plant with a C. lanatus plant;
b. selecting plants with watermelon fruits comprising an average fructose content of at least 50%; or sucrose content of at least 65%; or fructose and sucrose content of at least 90% of the total soluble sugar; and
c. selfing the selected plants at least once to obtain inbred line producing fruit with altered sugar ratios selected from at least one of elevated fructose and elevated sucrose content, having equal or reduced total sugar content, being devoid of bitterness and having superior sweet taste characteristics compared to currently available varieties and suitable for commercial scale cultivation.

78. The method of claim 77, wherein selfing is repeated 1 to 12 times.

79. A method of producing first generation hybrid seeds comprising crossing a first parent watermelon plant with a second parent watermelon plant and harvesting the resultant hybrid F₁ seeds, wherein the first and the second parent plants are inbred lines producing fruits with altered sugar ratios selected from at least one of elevated fructose and elevated sucrose content, having equal or reduced total sugar content, being devoid of bitterness and having superior sweet taste characteristics compared to currently available varieties, suitable for commercial scale cultivation.

80. A hybrid watermelon seed produced by the method of claim 79.

81. A hybrid watermelon plant, or parts thereof, produced by growing the seed of claim 80.

82. A method for producing a watermelon plant derived from the watermelon plant of claim 64, comprising:
a. crossing a first watermelon plant line with a second watermelon plant to obtain F₁ progeny seed, wherein the first watermelon plant is a plant according to claim 64;
b. growing the F₁ progeny seed under suitable plant growth conditions to yield an F₁ watermelon plant of the first hybrid plant; optionally;
c. crossing the plant obtained in step (b) with itself or with a third watermelon plant to yield second progeny seeds derived from said first hybrid plant; and
d. growing the second progeny seed under suitable plant growth conditions to yield additional watermelon plant derived of said first hybrid plant.

83. The method of claim 82, further comprising the step of repeating the steps of crossing the plant obtained in step (b) and growing the progeny seed at least 1 to 7 times to generate further watermelon plants.
